(19) United States
(12) Patent Application Publication
(10) Pub. No.: US 2026/0053956 A1
(43) Pub. Date: Feb. 26, 2026

(54) WATER-SOLUBLE CHLOROPHYLL-BINDING PROTEIN (WSCP)-CHLOROPHYLL COMPLEX AS CONTRAST AGENT FOR PHOTOACOUSTIC IMAGING

(71) Applicant: The Hong Kong University of Science and Technology, Hong Kong (CN)

(72) Inventors: Tsz Wai WONG, Hong Kong (CN); Tsz Chun Victor TSANG, Hong Kong (CN); Hayeon KIM, Hong Kong (CN)

(21) Appl. No.: 19/309,748

(22) Filed: Aug. 26, 2025

Related U.S. Application Data
(60) Provisional application No. 63/686,861, filed on Aug. 26, 2024.

(51) Int. Cl. A61K 49/22; A61B 5/00

(52) U.S. Cl. CPC A61K 49/221; A61B 5/0095

(57) ABSTRACT

Photoacoustic imaging (PAI) holds immense potential for non-invasive anatomical and functional imaging. Water-soluble chlorophyll-binding proteins (WSCPs) from Lepidium virginicum were successfully reconstituted with chlorophyll a, bacteriochlorophyll a, and bacteriochlorophyll b. The resulting complexes exhibit strong near-infrared (NIR) absorption, distinct and non-overlapping spectral profiles, and concentration-dependent photoacoustic (PA) signal generation. These properties make them suitable as contrast agents for PAI in various applications, particularly in clinical settings such as disease detection and monitoring.

### CROSS-REFERENCE TO RELATED APPLICATIONS

The present application claims priority to U.S. Provisional Patent Application No. 63/686,861, filed on Aug. 26, 2024. The contents of this application are hereby incorporated by reference in their entirety.

### TECHNICAL FIELD

The present disclosure relates to the use of water-soluble chlorophyll-binding protein (WSCP)-chlorophyll complex as contrast agent for photoacoustic imaging.

### BACKGROUND

Photoacoustic (PA) imaging (PAI) has emerged as a powerful non-invasive imaging modality, capitalizing the photoacoustic effect to visualize anatomical structures and provide functional information at deep imaging depths. This technique relies on the absorption of pulsed laser light by endogenous chromophores or exogenous contrast agents inside biological tissues, leading to transient thermoelastic expansion and the generation of ultrasonic waves. These waves are then detected and analyzed to reconstruct images based on the distribution of the absorbing molecules.

While PAI benefits from endogenous absorbers like hemoglobin and melanin, exogenous contrast agents offer enhanced sensitivity and specificity for targeted imaging applications. Biocompatibility, stability, and efficient clearance are crucial for clinical translation of exogenous contrast agents.

Various materials, including organic dyes, nanoparticles, and genetically encoded proteins, have been explored as PA contrast agents. However, limitations such as photobleaching, toxicity, and complex synthesis processes hinder their widespread application.

The development and identification of novel substances serving as safe and effective contrast agents are important for expanding the clinical applications of PAI.

### SUMMARY

The present disclosure provides a method for photoacoustic imaging a subject, the method comprising: (a) administering an effective amount of a water-soluble chlorophyll-binding protein (WSCP)-chlorophyll complex at a site of the subject, wherein the WSCP-chlorophyll complex comprises a Lepidium virginicum WSCP (LvP) and a chlorophyll selected from the group consisting of chlorophyll a, bacteriochlorophyll a, and bacteriochlorophyll b; (b) irradiating the subject with light thereby inducing a photoacoustic signal from the WSCP-chlorophyll complex; (c) detecting the photoacoustic signal; and (d) generating a photoacoustic image of the site based on the detected photoacoustic signal.

In certain embodiments, the light is pulsed light having a wavelength in the range of 650 nm to 900 nm.

In certain embodiments, the WSCP-chlorophyll complex exhibits a Qy absorption peak at a wavelength of the pulsed light in the range of about 665 nm to 850 nm and a Qx absorption peak at a wavelength of the pulsed light in the range of about 578 nm to 592 nm.

In certain embodiments, the WSCP-chlorophyll complex exhibits: a Qy absorption peak at a wavelength of the pulsed light of about 665 nm; a Qy absorption peak at a wavelength of the pulsed light of about 765 nm, and a Qx absorption peak at a wavelength of the pulsed light of about 578 nm; or a Qy absorption peak at a wavelength of the pulsed light of about 850 nm, and a Qx absorption peak at a wavelength of the pulsed light of about 592 nm.

In certain embodiments, the chlorophyll a is Spinacia oleracea chlorophyll a, the bacteriochlorophyll a is Rhodospirillum rubrum bacteriochlorophyll a, or the bacteriochlorophyll b is Blastochloris viridis bacteriochlorophyll b.

In certain embodiments, the method further comprises: (b1) irradiating the subject with a first pulsed light at a first wavelength to induce a first photoacoustic signal from the WSCP-chlorophyll complex; (b2) irradiating the subject with a second pulsed light at a second wavelength that is different from the first wavelength to induce a second photoacoustic signal from the WSCP-chlorophyll complex; (c1) detecting the first photoacoustic signal and the second photoacoustic signal; and (d1) generating a differential photoacoustic image of the site based on the detected first photoacoustic signal and the detected second photoacoustic signal.

In certain embodiments, the first and second wavelengths differs in a range from about 20-235 nm, or from about 20-50 nm; or wherein the WSCP-chlorophyll complex exhibits a Qy absorption peak at one of the first and second wavelengths, and a reduced or negligible optical absorption at the other of the first and second wavelengths.

In certain embodiments, the first and second wavelengths, or the second and first wavelengths, are: in the range of about 650 nm to 850 nm and about 685 nm to 900 nm, respectively; about 665 nm and about 685 nm, respectively; about 765 nm and about 800 nm, respectively; or about 850 nm and about 890 nm, respectively.

In certain embodiments, generating the differential photoacoustic image comprises subtracting the second photoacoustic signal from the first photoacoustic signal, or subtracting the first photoacoustic signal from the second photoacoustic signal.

In certain embodiments, the method further comprises: (b3) irradiating the subject with pulsed light prior to administration of the WSCP-chlorophyll complex to induce a background photoacoustic signal from endogenous chromophores at the site; (c3) detecting the background photoacoustic signal; and (d3) generating a differential photoacoustic image of the site based on the detected background photoacoustic signal and the photoacoustic signal detected after administration of the WSCP-chlorophyll complex.

In certain embodiments, the pulsed light used to iridate the subject before and after administration of the WSCP-chlorophyll complex is of the same wavelength; and wherein the wavelength is within a range of about 650-850 nm.

In certain embodiments, the wavelength is about 665 nm, about 765 nm, or about 850 nm.

In certain embodiments, generating the differential photoacoustic image comprises subtracting the background photoacoustic signal from the photoacoustic signal detected after administration of the WSCP-chlorophyll complex.

In certain embodiments, the method comprises: (a4) administering an effective amount of two or more water-soluble chlorophyll-binding protein (WSCP)-chlorophyll complexes at two or more target sites of the subject, wherein each WSCP-chlorophyll complexes comprise a Lepidium virginicum WSCP (LvP) and a chlorophyll selected from the group consisting of chlorophyll a, bacteriochlorophyll a, and bacteriochlorophyll b; (b4) irradiating the subject with pulsed light at two or more excitation wavelengths thereby inducing two or more photoacoustic signals from the respective WSCP-chlorophyll complexes; (c4) detecting photoacoustic signals; and (d4) generating a composite photoacoustic image that spatially resolves the different target sites based on the detected photoacoustic signals.

In certain embodiments, the two or more excitation wavelengths correspond to the absorption maxima of the respective WSCP-chlorophyll complexes and are in the range of about 665 nm to 850 nm.

In certain embodiments, the chlorophyll comprises any two or all of Spinacia oleracea chlorophyll a, Rhodospirillum rubrum bacteriochlorophyll a, and Blastochloris viridis bacteriochlorophyll b; or the excitation wavelengths comprise any two or all of the following: about 665 nm, about 765 nm, about 850 nm.

In certain embodiments, the subject is a human, a non-human primate, a rodent, a canine, a feline, a bovine, or an equine.

In another aspect, the present disclosure provides a method for photoacoustic imaging, the method comprising: providing an effective amount of a water-soluble chlorophyll-binding protein (WSCP)-chlorophyll complex comprising a Lepidium virginicum WSCP (LvP) and a chlorophyll selected from the group consisting of chlorophyll a, bacteriochlorophyll a, and bacteriochlorophyll b; irradiating the WSCP-chlorophyll complex with pulsed light having a wavelength in the range of 650 nm to 900 nm thereby inducing a photoacoustic signal; detecting the photoacoustic signal; and generating a photoacoustic image based on the detected photoacoustic signal.

In certain embodiments, the chlorophyll a is Spinacia oleracea chlorophyll a, the bacteriochlorophyll a is Rhodospirillum rubrum bacteriochlorophyll a, or the bacteriochlorophyll b is Blastochloris viridis bacteriochlorophyll b.

In a further aspect, the present disclosure provides a water-soluble chlorophyll-binding protein (WSCP)-chlorophyll complex, comprising a Lepidium virginicum WSCP (LvP) and Rhodospirillum rubrum bacteriochlorophyll a, or Blastochloris viridis bacteriochlorophyll b.

### BRIEF DESCRIPTION OF DRAWINGS

The above and other objects and features of the present disclosure will become apparent from the following description of the disclosure, when taken in conjunction with the accompanying drawings.

FIG. 1 shows a handheld linear array-based PAI system. (a) Schematic of the PAI system. (b) Photograph of the handheld array and fiber bundle setup. (c) Photograph of the AcousticX DAQ system. OPO, optical parametric oscillator; DAQ, data acquisition system.

FIG. 2 shows the result of size-exclusion chromatograms for (a) LvP-Chla, (b) LvP-Bchla, and (c) LvP-Bchlb solutions with normalized absorption for protein at 280 nm and corresponding Qy peaks.

FIG. 3 shows the absorption spectra of (a) LvP-chla, (b) LvP-bchla, and (c) LvP-bchlb as measured using UV-Vis spectroscopy. (d) Overlaid spectra of all three complexes for comparison of molar extinction coefficients to hemoglobin (dotted line) and oxyhemoglobin (solid line).

FIG. 4 shows spectroscopic characterization of LvP-chla. (a) UV-vis absorption spectra of LvP-chla (solid line) and naked LvP (dashed line), showing the characteristic chlorophyll a absorption peaks. (b) Molar extinction coefficient spectra of oxyhemoglobin (HbO₂, solid line) and deoxyhemoglobin (Hb, dashed line) in the visible to near-infrared region. (c) Excitation spectrum of LvP-chla monitored at 672 nm emission, revealing excitation maxima at 437 nm, 619 nm, and 663 nm. (d) Overlay of molar extinction coefficient spectra of LvP-chla, HbO₂, and Hb, demonstrating the superior absorption of LvP-chla at 665 nm compared to hemoglobin species.

FIG. 5 shows PA phantoms imaging with different excitation wavelengths in the NIR range (660 to 900 nm). (a) The plot of normalized PA amplitude for LvP complexes under different excitation wavelengths with moving average. (b) Collage of PA images of the phantoms with different excitation wavelengths with (c) PBS, (d) LvP-Chla, (e) LvP-Bchla, and (f) LvP-Bchlb solutions in corresponding glass capillary tubes.

FIG. 6 shows photoacoustic characterization of LvP-chla and blood phantoms. (a) Linear regression analysis showing normalized PA amplitude response of LvP-chla at different concentrations under 665 nm (upper dots) and 685 nm (lower dots) excitation, with blood controls (first diamond from top: whole blood 665 nm; second diamond from top: whole blood 685 nm). (b) Dual-modality imaging of phantom samples showing ultrasound image (top), PA image at 665 nm (middle), and PA image at 685 nm (bottom). Samples shown are (1) 0.5× blood, (2) whole blood, and LvP-chla at decreasing concentrations: (3) 10 mg/mL, (4) 7 mg/mL, (5) 5 mg/mL, (6) 3 mg/mL, and (7) 1 mg/mL. (c) Linear regression equations and R² values for normalized PA amplitude response of LvP-chla under 665 nm and 685 nm excitation. (d) Depth penetration study showing ultrasound image (left), PA image at 665 nm (middle), and overlay (right) of a LvP-chla phantom (5 mg/mL) embedded obliquely in chicken breast tissue. The dashed line boxes indicate the phantom position and white dashed line marks the tissue surface. (c) Quantitative analysis of PA signal strength versus depth from the tissue surface (vertical dotted line in right subfigure of figure (d)), showing imaging depth capability with the half-maximum signal at 6.30 mm depth.

FIG. 7 shows photoacoustic characterization of LvP-bchlb phantom. (a) Depth penetration study showing ultrasound image (left), PA image at 850 nm (middle), and overlay (right) of a LvP-bchlb phantom (5 mg/mL) embedded obliquely in chicken breast tissue. The dashed line boxes indicate the phantom position, white dashed line marks the tissue surface. (b) Quantitative analysis of PA signal strength versus depth from the tissue surface (vertical dotted line in right subfigure of figure (a)), showing imaging depth capability with the half-maximum signal at 12.13 mm depth.

FIG. 8 shows cytotoxicity of LvP-chla assessed by CyQUANT MTT Cell Viability Assay in 4T1 cells. Cells were treated with LvP-chla at 0, 1, 3, 5, 7, and 10 mg/mL for 12 h. Cell viability was quantified as a percentage relative to untreated control cells (0 mg/mL LvP-chla) after subtracting background absorbance (negative control: medium with MTT reagent, no cells). Data represent mean±standard deviation (n=4). Statistical analysis was performed using one-way ANOVA followed by Dunnett's post-hoc test (* p<0.05, ** p<0.01, *** p<0.001, and **** p<0.0001 versus positive control).

FIG. 9 shows experimental design and dual-modality imaging of LvP-chla delivery in a 4T1 tumor model. (a) Schematic workflow showing the experimental procedure: (1) subcutaneous injection of 4T1 cancer cells, (2) tumor development period, (3) initial PAI scanning, (4) intratumoral injection of LvP-chla, and (5) follow-up PAI experiment. Pre-injection imaging data (b-f): (b) Gross photograph of the tumor-bearing mouse, (c) ultrasound images showing tumor morphology in three orthogonal views, (d) PA images at 665 nm, (c) PA images at 685 nm, and (f) differential PA images (665 nm-685 nm). Post-injection imaging data (g-k): (g) Gross photograph after injection of 200 μL LvP-chla (5 mg/mL in PBS), (h) ultrasound images, (i) PA images at 665 nm, (j) PA images at 685 nm, and (k) differential PA images showing protein distribution. (1) Bar chart showing mean PA signals in the tumor region, averaged across xy, xz, and yz projections, before and after LvP-chla injection at 665 nm, 685 nm, and differential imaging. Dashed line circles indicate tumor boundaries. Scale bars: 10 mm for gross images.

FIG. 10 shows spatiotemporal analysis of differential PAI showing protein distribution and clearance over time. (a) xz-projection images before injection and at indicated time points (0, 24, 48, 72, and 96 h post-injection). (b) Normalized PA signals within regions of interest (dashed circle outlines at 0 h) were fitted to exponential decay models, yielding signal half-lives (t½) of 11.6 h (R2=1.000). Scale bars: 10 mm.

FIG. 11 shows H&E stained tissue sections from mice treated with LvP-chla. (a) Heart, showing right atrium valves (arrows); (b) Spleen, highlighting nominal white pulp (circle) and megakaryocytes (arrows); (c) 4T1 Tumor, demonstrating a necrotic region (circle); (d) Kidney, displaying a normal glomerulus; (c) Liver, exhibiting normal histological morphology.

FIG. 12 shows the photophysical characterization of the LvP-chla complex under various excitation conditions. (a-c) Fluorescence emission spectra following excitation at 437 nm, 619 nm, and 663 nm, respectively. (d) A comparative overlay of the emission profiles for all three excitation wavelengths in (a-c). (c) Key photophysical parameters of LvP-Chla. (f) Normalized absorbance and fluorescence spectra of LvP-Chla.

FIG. 13 shows the fluorescence and absorption characteristics of LvP-Bchla complex under various excitation conditions. (a-c) Fluorescence emission spectra following excitation at 357 nm, 577 nm, and 761 nm, respectively. (d) A comparative overlay of the emission profiles for all three excitation wavelengths in (a-c). (c) Normalized absorbance and fluorescence spectra of LvP-Bchla and LvP-chla, with indicated Stokes shifts of 11 nm and 28 nm, respectively. (f) Key photophysical parameters of LvP-Bchla.

### DETAILED DESCRIPTION

### Definitions

Throughout the present specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the present invention.

Furthermore, throughout the present specification and claims, unless the context requires otherwise, the word “include” or variations such as “includes” or “including”, will be understood to imply the inclusion of a stated integer or group of integers but not the exclusion of any other integer or group of integers.

The term “subject” as used herein, refers to an animal, typically a mammal or a human, that will be or has been the object of treatment, observation, and/or experiment. When the term is used in conjunction with the PAI described herein, then the subject has been the object of observation, monitor, and/or administration of the complex described herein.

The term “effective amount” as used herein, means that amount of the complex that generates a detectable acoustic signal upon irradiation with pulsed light in a cell culture, tissue system, subject, animal, or human.

The use of the singular herein includes the plural (and vice versa) unless specifically stated otherwise. In addition, where the use of the term “about” is before a quantitative value, the present teachings also include the specific quantitative value itself, unless specifically stated otherwise.

As used herein, the term “about” refers to a ±10%, ±9%, ±8%, ±7%, ±6%, ±5%, ±4%, ±3%, ±2%, ±1%, or ±0% variation from the nominal value unless otherwise indicated or inferred. The stated range includes the nominal value itself. For example, when referring to a range of “about 665 nm to 850 nm” in the claims or description, this range is intended to encompass variations such as “665 nm±5% to 850 nm±5%”, including ranges such as 660 nm to 855 nm, 665 nm to 855 nm, 670 nm to 855 nm, 660 nm to 845 nm, 670 nm to 845 nm, and so on. As another example, when referring to “about 665 nm”, “about 765 nm”, or “about 850 nm”, the recitation intends to encompass variations such as “about 665 nm±5% (including 660 nm, 665 nm, 670 nm, 661 nm, 664 nm, 667 nm, etc.)”, “about 765 nm±5% (including 760 nm, 765 nm, 770 nm, 761 nm, 764 nm, 767 nm, etc.)”, or “about 850 nm±5% (including 845 nm, 850 nm, 855 nm, 847 nm, 849 nm, 852 nm, 854 nm, etc.)”, and so on.

As used herein, the term “water-soluble chlorophyll-binding protein (WSCP)-chlorophyll complex (or WSCP-chlorophyll complex)” refers to a complex comprising WSCP and chlorophyll (such as chlorophyll a, chlorophyll b, bacteriochlorophyll a, bacteriochlorophyll b, or the like), wherein the WSCP and chlorophyll can be of different origins (e.g., from different parts of the same plant, from different individuals of the same species or from different species). Unless the context requires otherwise, the WSCP and chlorophyll will be understood to be independently obtained, either directly from an organism or being synthesized. For example, the WSCP-chlorophyll complex as described herein may include a Lepidium virginicum WSCP reconstituted with chlorophyll from a different plant or a bacterium, wherein the chlorophyll either is directly obtained from the plant or bacterium or synthesized in vitro.

As used herein, “irradiating the subject with light (thereby inducing a photoacoustic signal from the WSCP-chlorophyll complex)”, “irradiating the subject with pulsed light (such as a first/second pulsed light)”, or similar expressions, refer to an irradiation process performed at or near the surface of the subject to allow the light to arrive at a desired site, for example, a site where the WSCP-chlorophyll complex is administered or distributed, or intended to be administered or distributed. In certain instances where the irradiation is to be performed near the surface of the subject, the distance between the surface of the subject and the internal imaging location may range from about 0 mm to about 15 mm (such as about 1-13 mm, 2-12.5 mm, 3-9.5 mm, 4-9 mm, 4.5-8.5 mm, 5-8 mm, 5.5-7.5 mm, 6.3-13 mm, 6-12.5 mm, 6.2-12.2 mm, 6.5-11 mm, 7-11.5 mm, 7.5-10 mm or any individual values or subranges therebetween), depending on factors such as the specific device used, the intensity and wavelength of the light, and the like. In certain instances, the distance between the site of irradiation relative to the site of administration (in instances in which the complex is administered locally) may range from about 0 cm to about 15 cm (such as about 1-13 mm, 2-12.5 mm, 3-9.5 mm, 4-9 mm, 4.5-8.5 mm, 5-8 mm, 5.5-7.5 mm, 6.3-13 mm, 6-12.5 mm, 6.2-12.2 mm, 6.5-11 mm, 7-11.5 mm, 7.5-10 mm, or any individual values or subranges therebetween), depending on factors such as the specific device used, the intensity and wavelength of the light, and the like.

Similarly, “irradiating the WSCP-chlorophyll complex with pulsed light” or similar language may include the circumstances where the light source is positioned at a distance of about 0.1 cm to about 18 cm (such as about 0.1-5 cm, 0.5-3 cm, 2-3.5 cm, 1-4 cm, 1.5-2.5 cm, 1-12 cm, 2-10 cm, 3-8 cm, 4-7 cm, 5-15 cm, 6-13 cm, or any individual values or subranges therebetween) from the WSCP-chlorophyll complex or from a sample or container (such as a tube) comprising the WSCP-chlorophyll complex.

As used herein, “inducing a photoacoustic signal from the WSCP-chlorophyll complex”, “induce a first (or second) photoacoustic signal from the WSCP-chlorophyll complex”, or similar expressions may, in certain instances, encompass scenarios in which a background photoacoustic signal (e.g., photoacoustic signal generated by endogenous absorbers like chromophores, such as hemoglobin, and melanin) is also induced simultaneously during irradiation. In this context, the expression “detecting the photoacoustic signal” or similar language, may accordingly include detection of both the signal generated by the exogenous contrast agent (such as the WSCP-chlorophyll complex as described herein) and the background signal generated by the endogenous absorbers (if present). It will be appreciated that the influence of background signals may be mitigated or eliminated by means, such as differential photoacoustic imaging, or may be negligible in cases where the background signal is inherently weak relative to the signal generated by the exogenous contrast agent.

As used herein, “Qx” refers to the electronic transition dipole moment oriented along the short axis (x-diagonal) of the chlorin macrocycle, corresponding to the absorption peak at a relatively shorter wavelength; “Qy” refers to the electronic transition dipole moment along the long axis (y-diagonal), corresponding to the peak at a relatively longer wavelength, unless otherwise indicated.

As used herein, the term “differential photoacoustic image” refers to an image generated by comparing photoacoustic signals acquired under different conditions, such as (i) before and after administration of a contrast agent using pulsed light of the same wavelength, or (ii) after administration of a contrast agent using pulsed light of different wavelengths. This term may also include other imaging approaches that facilitate reduction or elimination of the influence of signals generated from endogenous absorbers, unless otherwise required.

In a first aspect, provided herein is a method for photoacoustic imaging a subject, the method comprising: (a) administering an effective amount of a water-soluble chlorophyll-binding protein (WSCP)-chlorophyll complex at a site of the subject; (b) irradiating the subject with light thereby inducing a photoacoustic signal from the WSCP-chlorophyll complex; (c) detecting the photoacoustic signal; and (d) generating a photoacoustic image of the site based on the detected photoacoustic signal.

In certain embodiments, the light is pulsed light, for example, pulsed light having a wavelength in the range of about 650 nm to 900 nm. In some embodiments, the wavelength is within a range of about 665 nm to 890 nm, such as about 665 nm, about 685 nm, about 700 nm, about 725 nm, about 750 nm, about 765 nm, about 764 nm, about 761 nm, about 800 nm, about 825 nm, about 850 nm, about 852 nm, about 890 nm, or any individual values or subranges therebetween.

In certain embodiments, the WSCP-chlorophyll complex can be excited at about 650-900 nm, or about 665-850 nm.

In certain embodiments, the WSCP-chlorophyll complex exhibits a Qy absorption peak at a wavelength of the pulsed light in the range of about 665 nm to 852 nm, such as about 665 nm to 850 nm, about 665 nm, about 761 nm, about 764 nm, about 765 nm, about 850 nm, or any individual values or subranges therebetween.

In certain embodiments, the WSCP-chlorophyll complex exhibits a Qx absorption peak at a wavelength of the pulsed light in the range of 578 nm to 592 nm, such as 578 nm, 592 nm, or any individual values or subranges therebetween.

In certain embodiments, the WSCP-chlorophyll complex exhibits: a Qy absorption peak at a wavelength of the pulsed light of about 665 nm; a Qy absorption peak at a wavelength of the pulsed light of about 761 nm, about 764 nm, or about 765 nm, and a Qx absorption peak at a wavelength of the pulsed light of 578 nm; or a Qy absorption peak at a wavelength of the pulsed light of about 850 nm, and a Qx absorption peak at a wavelength of the pulsed light of 592 nm.

In certain embodiments, the WSCP-chlorophyll complex comprises a Lepidium virginicum WSCP (LvP) and a chlorophyll, e.g., a LvP reconstituted with chlorophyll. In certain embodiments, the LvP is non-covalently conjugated to the chlorophyll. In certain embodiments, the chlorophyll is selected from the group consisting of chlorophyll a (e.g., S. oleracea chlorophyll a), bacteriochlorophyll a (e.g., R. rubrum bacteriochlorophyll a), and bacteriochlorophyll b (e.g., B. viridis bacteriochlorophyll b).

In certain embodiments, the method further comprises: (b1) irradiating the subject with a first pulsed light at a first wavelength to induce a first photoacoustic signal from the WSCP-chlorophyll complex; (b2) irradiating the subject with a second pulsed light at a second wavelength that is different from the first wavelength to induce a second photoacoustic signal from the WSCP-chlorophyll complex; (c1) detecting the first photoacoustic signal and the second photoacoustic signal; and (d1) generating a differential photoacoustic image of the site based on the detected first photoacoustic signal and the detected second photoacoustic signal.

In certain embodiments, the first and second wavelengths differs in a range from about 20 nm to 235 nm, or from about 20 nm to 50 nm. In certain embodiments, the first and second wavelengths differs in about 20 nm, 25 nm, 30 nm, 35 nm, 40 nm, 45 nm, 50 nm, 60 nm, 70 nm, 100 nm, 150 nm, or more.

In certain embodiments, the WSCP-chlorophyll complex exhibits a Qy absorption peak at one of the first and second wavelengths, and a reduced or negligible optical absorption at the other of the first and second wavelengths.

In certain embodiments, the first and second wavelengths, or the second and first wavelengths, are in the range of about 650 nm to 850 nm and about 685 nm to 900 nm, respectively. For example, the first and second wavelengths, or the second and first wavelengths, can be about 665 nm and about 685 nm (or 690 nm, 700 nm, 710 nm, 720 nm, 750 nm, or more), respectively; about 765 nm (or about 761 nm, about 764 nm) and about 800 nm (or 810 nm, 820 nm, 830 nm, 835 nm, 840 nm, 845 nm, or more), respectively; or, about 850 nm (or about 852 nm) and about 890 nm (or 900 nm), respectively. For example, the first and second wavelengths, or the second and first wavelengths, can respectively be: 665 nm and 690 nm; 665 nm and 700 nm; 665 nm and 710 nm; 665 nm and 720 nm; 665 nm and 750 nm.

In certain embodiments, step (d1) (i.e., generating the differential photoacoustic image) comprises subtracting the second photoacoustic signal from the first photoacoustic signal, or subtracting the first photoacoustic signal from the second photoacoustic signal.

In certain embodiments, the method further comprises: (b3) irradiating the subject with pulsed light prior to administration of the WSCP-chlorophyll complex to induce a background photoacoustic signal from endogenous chromophores at the site; (c3) detecting the background photoacoustic signal; and (d3) generating a differential photoacoustic image of the site based on the detected background photoacoustic signal and the photoacoustic signal detected after administration of the WSCP-chlorophyll complex. In certain embodiments, the pulsed light used to iridate the subject before and after administration of the WSCP-chlorophyll complex is of the same wavelength. In certain embodiments, the wavelength is within a range of 650-900 nm, such as about 650-850 nm, or about 665-850 nm. In certain embodiments, the wavelength is about 665 nm, about 765 nm, or about 850 nm.

In certain embodiments, the endogenous chromophores comprise naturally-occurring substances present at the site, such as hemoglobin, melanin, or any combination thereof.

In certain embodiments, step (d3) (i.e., generating the differential photoacoustic image) comprises subtracting the background photoacoustic signal from the photoacoustic signal detected after administration of the WSCP-chlorophyll complex.

In certain embodiments, the method comprises: (a4) administering an effective amount of two or more water-soluble chlorophyll-binding protein (WSCP)-chlorophyll complexes at two or more target sites of the subject, wherein each WSCP-chlorophyll complexes comprise a Lepidium virginicum WSCP (LvP) and a chlorophyll selected from the group consisting of chlorophyll a, bacteriochlorophyll a, and bacteriochlorophyll b; (b4) irradiating the subject with pulsed light at two or more excitation wavelengths thereby inducing two or more photoacoustic signals from the respective WSCP-chlorophyll complexes; (c4) detecting photoacoustic signals; and (d4) generating a composite photoacoustic image that spatially resolves the different target sites based on the detected photoacoustic signals.

In certain embodiments, the chlorophyll comprises any two or all of Spinacia oleracea chlorophyll a, Rhodospirillum rubrum bacteriochlorophyll a, and Blastochloris viridis bacteriochlorophyll b.

In certain embodiments, the method comprises administering two or three WSCP-chlorophyll complexes. In certain embodiments, the chlorophyll is selected from the group consisting of chlorophyll a (e.g., S. oleracea chlorophyll a), bacteriochlorophyll a (e.g., R. rubrum bacteriochlorophyll a), and bacteriochlorophyll b (e.g., B. viridis bacteriochlorophyll b). In instances where two WSCP-chlorophyll complexes are administered, one WSCP-chlorophyll complex comprises S. oleracea chlorophyll a, and the other comprises either of R. rubrum bacteriochlorophyll a, and B. viridis bacteriochlorophyll b. In instances where three or more WSCP-chlorophyll complexes are administered, three of the WSCP-chlorophyll complexes respectively comprises S. oleracea chlorophyll a, R. rubrum bacteriochlorophyll a, and B. viridis bacteriochlorophyll b.

In certain embodiments, two or more WSCP-chlorophyll complexes are administered to the same target site. Alternatively, each of the complexes may be administered to a distinct target site.

In certain embodiments, the two or more excitation wavelengths correspond to the absorption maxima of the respective WSCP-chlorophyll complexes. In certain embodiments, the two or more excitation wavelengths are in the range of about 665 nm to 850 nm. In certain embodiments, the two or more excitation wavelengths are spectrally distinct. In certain embodiments, the two or more excitation wavelengths comprise any two of the following: about 665 nm, about 765 nm, about 850 nm. In instances where three or more WSCP-chlorophyll complexes are administered, the excitation wavelengths comprise all of the following: about 665 nm, about 765 nm, about 850 nm.

In certain embodiments, the site is selected from the group consisting of blood, tissue, urine, tumor, kidney, heart, spleen, liver, lung, pancreas, brain, or lymph node, a cancer site, any other diseased site within the subject, or any combination thereof. Examples of cancer or tumor may include superficial and subcutaneous cancers (such as cervical cancer, skin cancer, thyroid cancer, and malignant brain tumors), breast cancer, prostate cancer, kidney cancer, liver cancer, pancreas cancer, lung cancer, colorectal cancer, melanoma, bladder cancer, non-Hodgkin lymphoma, endometrial cancer, pancreatic cancer, leukemia, liver cancer, or any combination thereof.

In certain embodiments, the WSCP-chlorophyll complex is administered intravenously, subcutaneously, intraperitoneally, intramuscularly, intratumorally, intraarterially, topically, or orally. In certain embodiments, the WSCP-chlorophyll complex is administered subcutaneously.

In certain embodiments, the WSCP-chlorophyll complex is included in or formulated into a composition (e.g., a pharmaceutical composition). In such instances, the composition may be in the form of an aqueous solution, dispersion, or suspension, and may comprise a pharmaceutical acceptable carrier. Examples of the carriers include physiological saline, bacteriostatic water, or phosphate-buffered saline (PBS).

In certain embodiments, the WSCP-chlorophyll complex is used (such as administered) in a concentration of about 1-10 mg/mL, such as about 1-7 mg/mL, about 2-6 mg/mL, about 3-8 mg/mL, about 4-5 mg/mL, about 1-5 mg/mL, e.g., about 3 mg/mL, 4 mg/mL, 5 mg/mL, or any individual values or subranges therebetween. In instances where the WSCP-chlorophyll complex is included in or formulated into a composition, the composition may comprise the WSCP-chlorophyll complex in a concentration of about 1-10 mg/mL, such as about 1-7 mg/mL, about 2-6 mg/mL, about 3-8 mg/mL, about 4-5 mg/mL, about 1-5 mg/mL, e.g., about 3 mg/mL, 4 mg/mL, 5 mg/mL, 5.5 mg/mL, 6 mg/mL, or any individual values or subranges therebetween.

In certain embodiments, the method described herein achieves a detectable photoacoustic signal at depths of up to about 12.5 mm (such as about 12.5 mm, 12.13 mm, 6.30 mm) in scattering media, with the signal intensity reaching half of its maximum at this depth. In such embodiments, scattering media may refer to optically turbid materials that mimic the light-scattering properties of biological tissues. Scattering media may include both ex vivo and in vivo biological tissues, as well as synthetic phantoms designed to simulate such environments. Examples of materials suitable for scattering media may include intralipid solutions (e.g., 1-2%), agarose or gelatin-based phantoms, latex or polystyrene microspheres, titanium dioxide (TiO₂), ex vivo biological tissues (e.g., chicken breast), in vivo tissues, custom tissue-mimicking phantom, or the like.

In certain embodiments, the method described herein enables detection of photoacoustic signals generated by WSCP-chlorophyll complexes located at depths of up to about 12.5 mm (such as about 12.5 mm, 12.13 mm, 6.30 mm) beneath the surface of the subject.

In certain embodiments, the WSCP-chlorophyll complex comprises a LvP having four subunits, wherein each subunit is capable of binding at most one chlorophyll molecule. In certain embodiments, each subunit binds with a chlorophyll molecule of the same type, if present. In certain embodiments, a single WSCP-chlorophyll complex comprises one, two, three, or four chlorophyll molecules of the same type.

In certain embodiments, the subject is a human, a non-human primate, a rodent, a canine, a feline, a bovine, or an equine.

In a second aspect, provided herein is a method for photoacoustic imaging, the method comprising: providing an effective amount of a water-soluble chlorophyll-binding protein (WSCP)-chlorophyll complex; irradiating the WSCP-chlorophyll complex with light thereby inducing a photoacoustic signal; detecting the photoacoustic signal; and generating a photoacoustic image based on the detected photoacoustic signal. The method may be conducted in vitro or in vivo.

In certain embodiments, the light is pulsed light, for example, pulsed light having a wavelength in the range of about 650 nm to 900 nm. In some embodiments, the wavelength is within a range of about 665 nm to 890 nm, such as about 665 nm, about 685 nm, about 700 nm, about 725 nm, about 750 nm, about 765 nm, about 764 nm, about 761 nm, about 800 nm, about 825 nm, about 850 nm, about 852 nm, about 890 nm, or any individual values or subranges therebetween.

In certain embodiments, the WSCP-chlorophyll complex is as described in any embodiment disclosed herein.

In certain embodiments, the WSCP-chlorophyll complex comprises a Lepidium virginicum WSCP (LvP) and a chlorophyll selected from the group consisting of chlorophyll a (e.g., S. oleracea chlorophyll a), bacteriochlorophyll a (e.g., R. rubrum bacteriochlorophyll a), and bacteriochlorophyll b (e.g., B. viridis bacteriochlorophyll b).

In certain embodiments, the WSCP-chlorophyll complex is included in or formulated into a composition (e.g., a pharmaceutical composition). In such instances, the composition may be in the form of an aqueous solution, dispersion, or suspension, and may comprise a pharmaceutical acceptable carrier. Examples of the carriers include physiological saline, bacteriostatic water, or phosphate-buffered saline (PBS). In certain embodiments, the WSCP-chlorophyll complex is contained in a container, such as a tube.

In certain embodiments, the method further comprises irradiating the WSCP-chlorophyll complex with a first pulsed light at a first wavelength to induce a first photoacoustic signal; irradiating the WSCP-chlorophyll complex with a second pulsed light at a second wavelength to induce a second photoacoustic signal; detecting the first and second photoacoustic signal; and generating a photoacoustic image based on the detected first and second photoacoustic signal. In certain embodiments, generating the differential photoacoustic image comprises subtracting the second photoacoustic signal from the first photoacoustic signal, or subtracting the first photoacoustic signal from the second photoacoustic signal. In certain embodiments, the first and second wavelengths are as defined in the first aspect of the present disclosure.

In a third aspect, provided herein is a water-soluble chlorophyll-binding protein (WSCP)-chlorophyll complex.

In certain embodiments, the WSCP-chlorophyll complex is as defined in any embodiment disclosed herein. In certain embodiments, the WSCP-chlorophyll complex comprises a Lepidium virginicum WSCP (LvP) and Rhodospirillum rubrum bacteriochlorophyll a, or Blastochloris viridis bacteriochlorophyll b.

In a fourth aspect, provided herein is a use of the WSCP-chlorophyll complex as defined in any embodiment disclosed herein as a contrast agent. The contrast agent is useful in imaging a subject (e.g., imaging the disease of the subject), monitoring the disease/condition of the subject, or diagnosing if the subject has a disease. In certain embodiments, the disease includes a cancer or a tumor. Examples of cancer or tumor may include superficial and subcutaneous cancers (such as cervical cancer, skin cancer, thyroid cancer, and malignant brain tumors), breast cancer, prostate cancer, kidney cancer, liver cancer, pancreas cancer, lung cancer, colorectal cancer, melanoma, bladder cancer, non-Hodgkin lymphoma, endometrial cancer, pancreatic cancer, leukemia, liver cancer, or any combination thereof.

In a fifth aspect, provided herein is a use of the WSCP-chlorophyll complex as defined in any aspect of the present disclosure in the manufacture of a contrast agent. In certain embodiments, the contrast agent is used in the method as defined in the first and/or second aspect of the present disclosure.

In certain embodiments of any of the aspects of the present disclosure, the WSCP-chlorophyll complex further comprises a targeting moiety to facilitate targeted delivery. In certain embodiments, the targeting moiety is selected from the group consisting of a protein, a peptide, an antibody or antigen-binding fragment thereof, a ligand, a nucleic acid aptamer, and any combination thereof. The targeting moiety can be covalently conjugated directly to the WSCP-chlorophyll complex (e.g., to the WSCP or the chlorophyll) or linked to the WSCP-chlorophyll complex via a linker.

In certain embodiments of any of the aspects of the present disclosure, the pulsed light is generated by a laser, a light-emitting diode, a laser diode, a flash lamp, or any combination thereof.

In certain embodiments of any of the aspects of the present disclosure, the method further comprises displaying the photoacoustic image on a user interface (such as a computer).

The present disclosure explores the potential of water-soluble chlorophyll-binding proteins (WSCPs) as a novel class of PA contrast agents. The present disclosure successfully reconstituted a recombinant WSCP originated from Lepidium virginicum (LvP), with chlorophyll a sourced from Spinacia oleracea, bacteriochlorophyll a from Rhodospirillum rubrum, or bacteriochlorophyll b from Blastochloris viridis. The reconstituted WSCPs (i.e., the complexes of the present disclosure) exhibit strong NIR absorption due to their ability to bind and stabilize chlorophyll and bacteriochlorophyll molecules.

As demonstrated in the following examples, the reconstituted complexes exhibit strong near-infrared (NIR) absorption with a high molar extinction coefficient and a narrow Qy absorption band, enabling deep tissue penetration and minimal interference from endogenous chromophores. The complexes show concentration-dependent signal generation, enhanced imaging contrast, and effective tumor site visualization in both phantom and in vivo experiments. These findings establish the complexes as efficient and ideal contrast agents for PAI, offering strong potential for clinical translation in non-invasive anatomical and functional imaging.

Also, toxicity evaluations of the WSCP-chlorophyll complexes reveal excellent biocompatibility, with no observable toxicity or adverse effects, along with efficient systemic clearance—underscoring their biosafety for in vivo applications.

The WSCP-chlorophyll complexes described herein enable detection of photoacoustic signals at depths of up to about 12.5 mm (such as about 12.5 mm, 12.13 mm, 6.30 mm) beneath the surface of the subject. In certain embodiments, the detectable depth includes depths such as approximately 6.30 mm or 12.13 mm. Surprisingly, LvP-Bchlb can achieve an imaging depth of 12.13 mm in tissue-mimicking phantoms, which is nearly double the 6.30 mm depth achieved by LvP-Chla under identical conditions. This improvement in penetration depth is dramatic, and represents a significant technical advantage of LvP-Bchlb.

Spectroscopic characterization reveals that LvP-Bchla exhibits a photoluminescence quantum yields (PLQY) of only 3.8%, representing a substantial 49% reduction compared to the 7.5% PLQY measured for LvP-Chla. This lower quantum yield is particularly beneficial for photoacoustic signal generation, as it indicates that a greater proportion of absorbed photon energy is converted to heat, thereby enhancing photoacoustic signal generation. The protein scaffold appears to induce different quenching effects depending on the specific pigment, with bacteriochlorophylls experiencing enhanced non-radiative relaxation compared to chlorophyll a. This effect is even more pronounced when considering that free bacteriochlorophyll typically exhibits quantum yields of 16-20% in organic solvents, while free chlorophyll a shows approximately 29% PLQY in ethanol. The dramatic reduction in quantum yield for bacteriochlorophyll variants among the present complexes represents an unexpected synergistic effect. This enhanced thermal conversion efficiency, combined with the red-shifted absorption profiles, contributes to the superior photoacoustic performance of the bacteriochlorophyll complexes, enabling stronger signal generation at greater tissue depths.

Collectively, these results underscore the promise of the present complexes as a safe, stable, effective, and reliable PA contrast agent with strong potential for clinical translation.

Following are examples that illustrate procedures for practicing the invention. These examples should not be construed as limiting. All percentages are by weight and all solvent mixture proportions are by volume unless otherwise noted.

### EXAMPLES

### Materials and Methods

### 1. Contrast Agent Synthesis

### 1.1. Gene Construction and Expression of LvP

The gene encoding the WSCP from LvP (PDB code: 6GIW), reported by Palm et al. (Palm, D. M.; Agostini, A.; Averesch, V.; Girr, P.; Werwie, M.; Takahashi, S.; Satoh, H.; Jaenicke, E.; Paulsen, H. Chlorophyll a/b binding-specificity in water-soluble chlorophyll protein. Nat. Plants 2018, 4, 920-929), was synthesized and cloned into pET22b expression vector. Escherichia coli strain BL21 (DE3) carrying the pET22b::WSCP construct was cultured in Luria-Bertani (LB) medium supplemented with 100 mg/L ampicillin at 37° C. until the optical density at 600 nm (OD600) reached 0.6-1.2. Protein expression was then induced by adding 400 μM Isopropyl β-D-1-thiogalactopyranoside (IPTG) and incubated at 37° C. for 3 hours.

1.2. Reconstitution LvP with Photosynthetic Pigments

Photosynthetic pigment chlorophyll a was reconstituted into LvP based on methods reported by Li et al. (Li, M.; Park, B. M.; Dai, X.; Xu, Y.; Huang, J.; Sun, F. Controlling synthetic membraneless organelles by a red-light-dependent singlet oxygen-generating protein. Nat. Commun. 2022, 13, 3197). Fresh spinach leaves were used as the source of chlorophyll a (chla). The induced E. coli pellets containing LvP were homogenized with spinach leaves at a mass ratio of about 5:1 (spinach: E. coli pellet) along with the lysis buffer [containing 300 mM of sodium chloride, 20 mM Tris buffer and 0.1 mM PMSF (Phenylmethylsulfonyl fluoride), pH7.5]. The mixture was then sonicated with a Branson sonicator at 150 W power with 0.5 s intervals for 30 s followed by 60 s rest. The sonication was done in 2 rounds, 5 minutes each, for a total of 10 minutes.

For bacteriochlorophylls, bacteriochlorophyll-containing phototropic bacteria R. rubrum and B. viridis were respectively cultured in sodium succinate medium, as previously described by Qian et al. (P. Qian, C. A. Siebert, P. Wang, D. P. Canniffe, and C. N. Hunter, “Cryo-EM structure of the Blastochloris viridis LH1-RC complex at 2.9 Å,” Nature, vol. 556, no. 7700, pp. 203-208, April 2018. doi: 10.1038/s41586-018-0014-5). The cell pellets were then sonicated with the Lvp-expressing E. coli pellets in lysis buffer, ensuring thorough mixing and reconstitution of Lvp with bacteriochlorophylls (bacteriochlorophyll a, i.e., bchla; or bacteriochlorophyll b, i.e., bchlb). This straightforward approach facilitates the creation of the desired contrast agent for photoacoustic imaging tailored to the specific chlorophyll type being utilized.

### 1.3. Protein Purification:

After sonication, the resulting homogenates were centrifuged at 14,000×g for 30 min at 4° C. to separate the supernatants containing the reconstituted protein (i.e., Lvp-chla, Lvp-bchla, or Lvp-chlb) from the insoluble fractions. The soluble reconstituted protein was then purified using standard nickel-nitrilotriacetic acid (Ni-NTA) affinity chromatography with HisTrap™ HP columns (Cytiva, Marlborough, MA, USA). The purification process involves the use of a wash buffer (300 mM NaCl, 20 mM Tris, 25 mM imidazole, pH 7.5) to remove impurities and an elution buffer (300 mM NaCl, 20 mM Tris, 500 mM imidazole, pH 7.5) to elute the purified protein.

Size exclusion chromatography (SEC) was performed as an additional purification step to refine the protein preparation further. The protein purity was assessed using SDS-PAGE analysis. After SEC, the eluted protein fractions were analyzed by UV-vis spectroscopy to confirm the successful reconstitution of the protein-pigment complexes.

The obtained WSCP contrast agent can be used immediately or stored at −80° C. after flash-freezing in liquid nitrogen for future applications in photoacoustic. The final reconstituted protein solution was prepared in phosphate-buffered saline (PBS) for injection.

### 1.4. Spectral Measurement

The absorption spectrum of the reconstituted protein solution, along with oxyhemoglobin (HbO₂), deoxyhemoglobin (Hb), and naked LvP (without chla, bchla, or bchlb), was measured with a Varioskan LUX multimode microplate reader with PBS as the blank reference. Spectral scans were performed over a wavelength range from 300 nm to 900 nm with 1 nm resolution. After that, the concentration of the protein solution was determined by a NanoDrop spectrophotometer (Thermo Fisher Scientific, Waltham, MA, USA). The molar extinction coefficient was then calculated using the Beer-Lambert law with the normalized absorbance and the concentration.

### 1.5. Photoluminescence Quantum Yield (PLQY) Measurement

The PLQY was recorded on an ultraviolet to NIR absolute PLQY spectrometer (Quantaurus-QY, Hamamatsu Photonics K.K., Shizuoka, Japan) at room temperature. This instrument utilizes an integrating sphere with a fluorometer to measure the ratio of emitted photons to absorbed photons. The process involved exciting the sample, measuring the absorbed light, and collecting all emitted photons for quantification. The quantum yield was then calculated directly, providing values that indicate the efficiency of light conversion into fluorescence versus heat.

### 2. PAI Setup

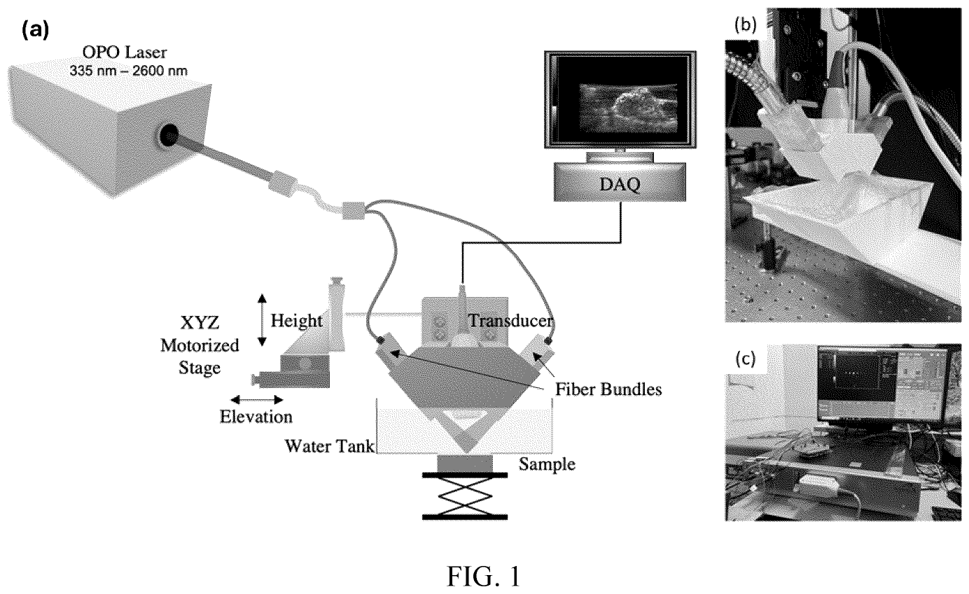

The PAI system employed in this study is based on the configuration described by Wang et al. (Wang, S.; Huang, B.; Chan, S. C.; Tsang, V. T.; Wong, T. T. Tri-modality in vivo imaging for tumor detection with combined ultrasound, photoacoustic, and photoacoustic elastography. Photoacoustics 2024, 38, 100630). Minor modifications were implemented to adopt the system for small animal imaging (FIG. 1a). The imaging setup consists of a linear array ultrasound transducer (UST) positioned coaxially with the optical fiber bundle using a custom-made 3D-printed adapter (FIG. 1b) within a water tank. An optically transparent membrane was adhered to the bottom of the water tank to facilitate effective coupling of the generated PA signals. During operation, the tumor-bearing mouse can be placed in a supine position. Ultrasound gel can be applied directly to the skin of the nude mouse to enhance acoustic coupling between the tissue and the UT. The optical fiber bundle delivers pulsed laser excitation from an optical parametric oscillator (OPO; NT352C-20-FWS-SH-H, EKSPLA Inc., Vilnius, Lithuania) to the imaging region. Data acquisition was managed by a commercial system (AcousticX, Cyberdyne Inc., Tsukuba, Japan) connected to a personal computer (PC), as shown in FIG. 1c.

### 3. PA Phantom Imaging

The chlorophyll-reconstituted WSCP proteins were serially diluted to create a concentration gradient from 1 mg/mL to 10 mg/mL and carefully loaded into thin capillary tubes via capillary action to create phantom samples for PAI in order to obtain phantom images using PAI. Additionally, 1 mL of mouse whole blood was harvested via cardiac puncture, and 0.1 mL of ethylenediamine tetraacetic acid solution was added to prevent coagulation. A 0.5× diluted blood sample was prepared by mixing 0.5 mL of whole blood with 0.5 mL of PBS and loaded into separate capillary tubes. The loaded capillary tubes are sealed with glue or over an open flame to ensure the containment of the samples. The prepared capillary tubes were placed on a holder or stage, ensuring stability during imaging. Next, the capillary tubes with the WSCP contrast agents were positioned within the imaging field, allowing for the acquisition of photoacoustic signals generated by the contrast agent and blood samples under laser excitation.

The imaging process was performed using the setup shown in FIG. 1. The imaging system is configured to emit light pulses at a specific wavelength using an OPO laser, and the photoacoustic signals are detected by a 7 MHz linear array ultrasound transducer in the AcousticX system. The built-in software of the AcousticX system generates the final PA images.

### 4. Animal Imaging

### 4.1. Tumor Mouse Model

Nu/J mice (The Jackson Laboratory, Bar Harbor, ME, USA), aged 4-5 weeks, were utilized to establish 4T1 cell line allografts. Each mouse received subcutaneous injections of 1×10⁶ 4T1 cells suspended in 100 μL PBS at two sites: the right hindlimb and the left forelimb. Mice were housed under standard conditions (temperature: ˜ 22° C., humidity: 40-70%, 12 h light/dark cycles) with free access to sterile food and water. Tumor growth was monitored regularly, and imaging experiments were conducted once tumor volumes reached approximately 2000 mm³. All experiments were carried out in conformity with a laboratory animal protocol approved by the Health, Safety, and Environment Office of The Hong Kong University of Science and Technology (Approval Number: AEP-2022-0010).

### 4.2. Image Acquisition

The tumor-bearing mice were sedated via intraperitoneal injection of a ketamine/xylazine/saline (KXS) cocktail composed of 17.5% v/v ketamine, 2.5% v/v xylazine, and 80% v/v sterile saline at a dosage of 5 μL/g body weight. The plane of anesthesia was confirmed by performing a toe-pinch reflex test. The mouse was then positioned in a supine orientation on the imaging stage. A total of 200 μL of 5 mg/mL LvP-chla solution was administered via intratumoral injection. Ultrasound gel was applied to the tumor site and surrounding region to facilitate acoustic coupling. PAI was performed using a laser wavelength of 665 nm, corresponding to the absorption maximum of LvP-chla, to acquire volumetric PA images along the midline towards the caudal direction. Subsequently, the same volumetric scan was repeated using a 685 nm laser excitation, at which the molar extinction coefficient of LvP-chla is reduced by half. The laser operated at a repetition rate of 20 Hz with pulse widths ranging from 3 to 5 ns. The laser fluence on the tissue surface was approximately 16.1 mJ/cm² for 665 nm and 15.6 mJ/cm² for 685 nm, both below the American National Standards Institute safety limit of 20 mJ/cm².

### 5. Toxicity Test

### 5.1. Cytotoxicity Assay

The cytotoxicity of LvP-chla was assessed using the CyQUANT MTT Cell Viability Assay kit (Thermo Fisher Scientific, Waltham, MA, USA) with 4T1 cells. The 4T1 cells were seeded at a density of 3×10⁴ cells per well in 96-well plates and cultured in RPMI-1640 medium supplemented with 10% fetal bovine serum and 1% penicillin-streptomycin at 37° C. in a 5% CO₂ atmosphere. After 24 h, cells were treated with fresh medium mixed with LvP-chla at concentrations of 0, 1, 3, 5, 7, and 10 mg/mL in 4 replicates. A negative control (medium with MTT reagent without cells) and a positive control (untreated cells, 0 mg/mL LvP-chla) were included. After 12 h of incubation, 10 μL of 12 mM MTT reagent was added to each well, and the plates were incubated for 4 h at 37° C. The resulting formazan crystals were solubilized by adding 100 μL of the kit's SDS-HCl solution (0.1% SDS in 0.01 M HCl) to each well, then incubating for 10 min at 37° C. with gentle mixing.

Absorbance was measured at 570 nm using a Varioskan LUX multimode microplate reader (Thermo Fisher Scientific, Waltham, MA, USA). Cell viability was calculated as a percentage relative to the positive control after subtracting the negative control absorbance. Data were analyzed using one-way ANOVA with Dunnett's post-hoc test to compare treated groups to the positive control, with significance set at p<0.05. GraphPad Prism (version 9.0) was used for statistical analysis.

### 5.2. Histology Study

To evaluate the potential cytotoxicity of LvP-chla in vivo, key organs (e.g., liver and kidney) and tumor tissues were harvested from euthanized mice following 96 h postinjection of 5 mg/mL LvP-chla. Euthanasia was performed by overdose of the KXS cocktail as described above. Harvested tissues were immediately fixed in 4% neutral-buffered formalin at room temperature for 24 h. After fixation, tissues were processed using a tissue processor (Revos, Thermo Fisher Scientific, Waltham, MA, USA) for 12 h, followed by paraffin embedding. Paraffin-embedded tissues were sectioned at a thickness of 5 μm using a microtome (RM2235, Leica Microsystems, Wetzlar, Germany) and mounted onto glass slides. The tissue sections were stained with hematoxylin and eosin (H&E), and the stained slides were imaged using a digital slide scanner (NanoZoomer-SQ, Hamamatsu Photonics K.K., Shizuoka, Japan).

### Results

### 1. Spectral Characterization of Reconstituted LvP Complexes

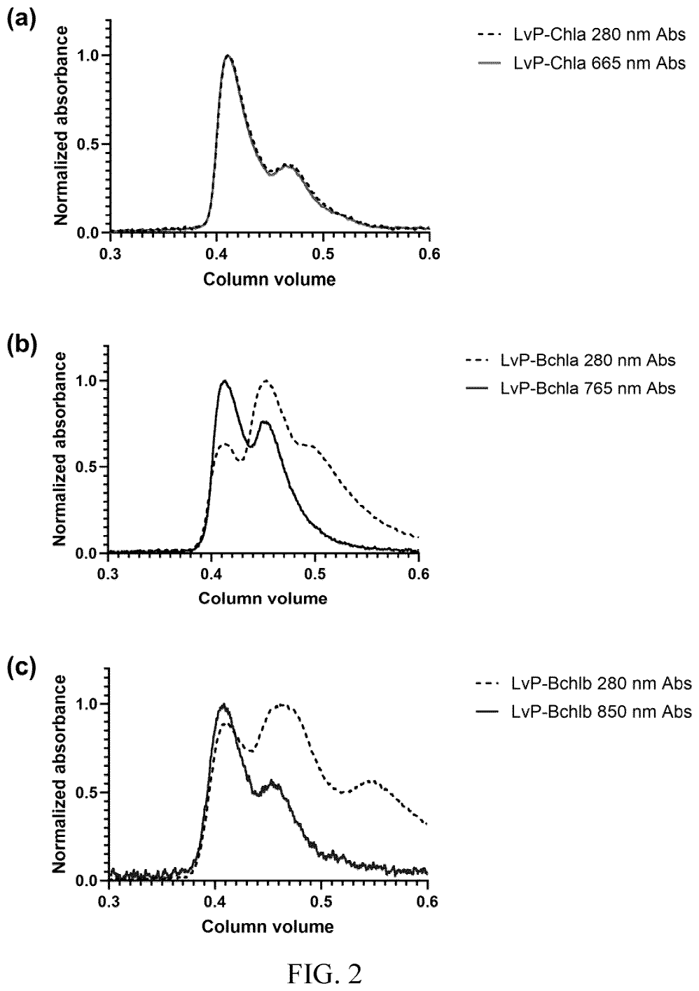

UV-vis spectroscopy confirmed the successful reconstitution of the three LvP complexes by detecting characteristic chlorophyll absorbance peaks (see FIG. 2). LvP-chla tends to form a homotetramer with pigments facing inwards in a hydrophobic pocket, preventing oxidative photobleaching when it's excited by optical absorption. A similar homotetramer formation with size-exclusion chromatography in the bacteriochlorophyll variants, as shown in FIG. 2, was observed, hence properties such as photobleaching resistance and stability in physiological temperature reported in LvP-chla are likely to be preserved in bacteriochlorophyll variants.

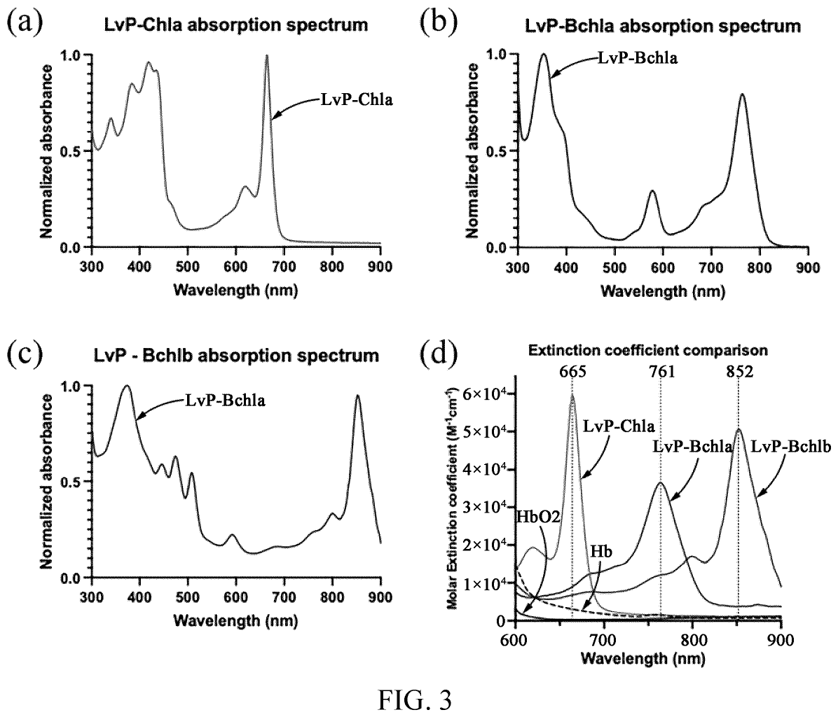

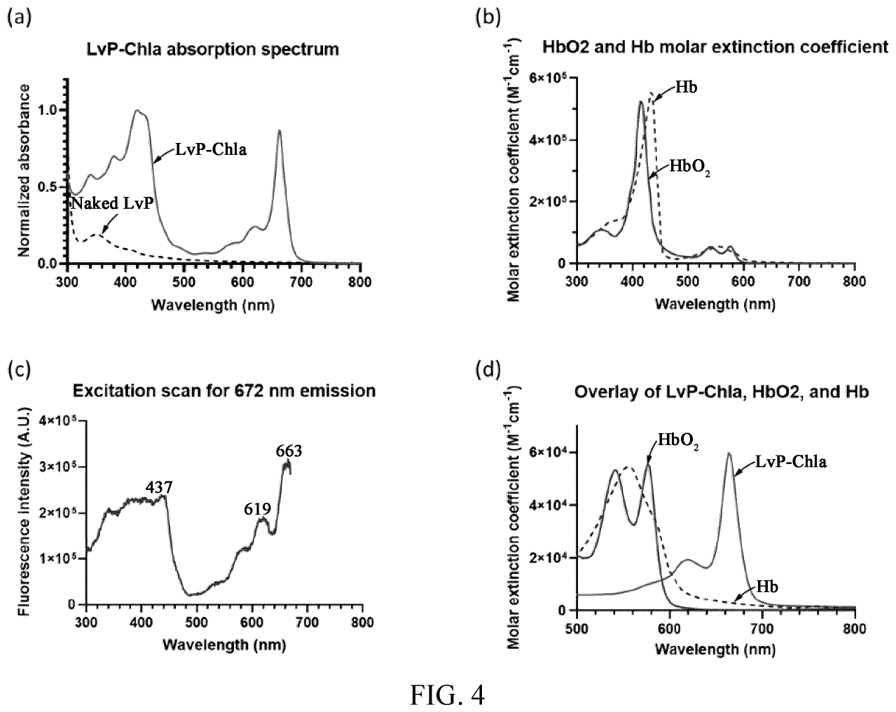

The absorption spectra of the reconstituted LvP complexes (LvP-chla, LvP-bchla, and LvP-bchlb), along with HbO₂, Hb, and naked LvP, were measured using UV-Vis spectroscopy (FIGS. 3-4). LvP-chla exhibited a strong Qy peak at 665 nm, characteristic of chlorophyll a (FIG. 3a, 4a), with a molar extinction coefficient (ε) of 59,716 M⁻¹ cm⁻¹. This value is significantly higher than that of Hb and HbO₂ at the same wavelength, as shown in the comparative spectra (FIG. 4b,d). The absorbance at 653 nm and 685 nm is reduced by half, resulting in a narrow Qy band with a full width at half maximum of 20 nm. The excitation spectrum monitored at 672 nm emission (FIG. 4c) reveals multiple excitation peaks, with maxima at 437 nm, 619 nm, and 663 nm.

LvP-bchla displayed a Qy peak at 765 nm and Qx at 578 nm, while LvP-bchlb showed a Qy peak at 850 nm and Qx peak at 592 nm (FIGS. 3b-c). The huge separation of the Qy, Qx peaks in bacteriochlorophylls reflects the structure differences thereof, resulting in more prominent excitation energy differences when compared to chlorophyll a. These distinct spectral characteristics, particularly for the bacteriochlorophyll-reconstituted complexes, demonstrate the successful binding of LvP with pigments beyond its natural counterparts and highlight their potential for multi-wavelength PAI.

Moreover, the higher absorption coefficients of these complexes compared to abundant endogenous contrasts such as hemoglobin and deoxyhemoglobins are noteworthy (FIG. 3d). The higher extinction coefficients and the Qy peaks within the far-red to near-infrared range, as the biological optical window, suggest that these complexes have significant potential as exogenous contrast agents for in vivo imaging.

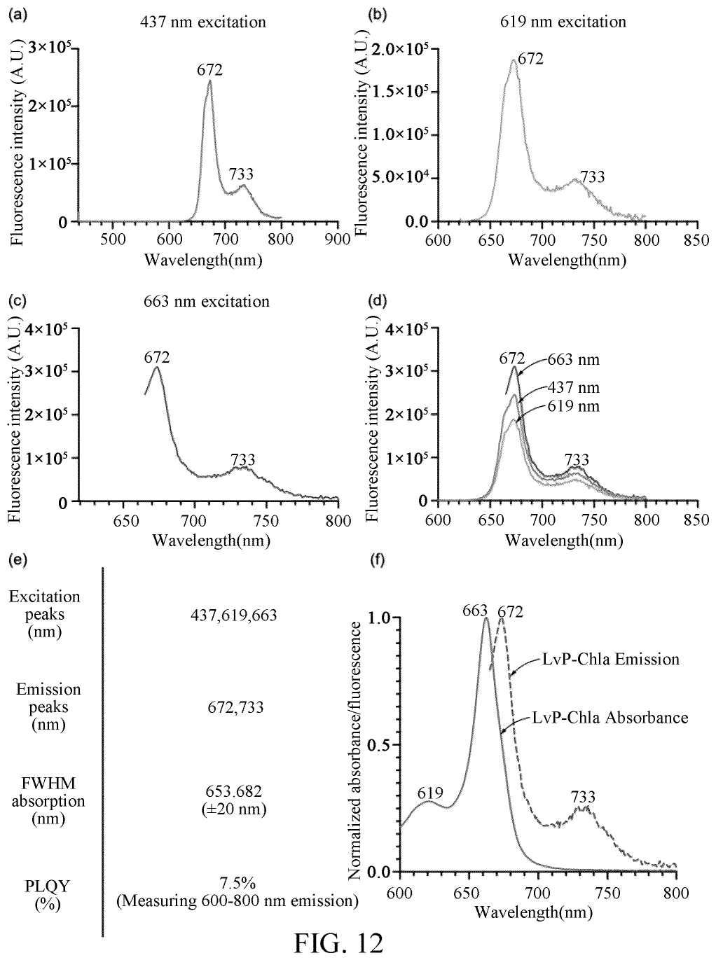

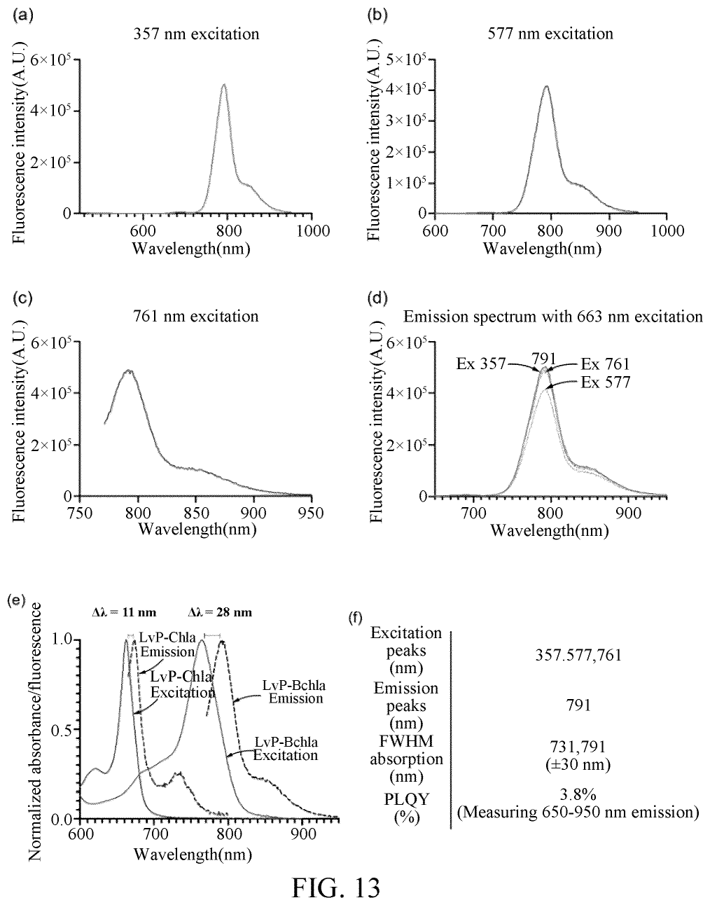

The PLQY was measured at 7.5% for LvP-chla, and 3.8% for LvP-bchla, demonstrating the enhanced thermal conversion efficiency of the bacteriochlorophyll complex that contributes to its superior photoacoustic performance (FIGS. 12-13). However, due to the extreme red-shift of its absorption, the corresponding fluorescence emission peak was predicted to lie beyond the detection range of the available spectrometer. Based on the consistent 28 nm Stokes shift observed for the LvP-bchla complex, it can be reasonably extrapolated that the fluorescence emission maximum for LvP-bchlb would occur at approximately 878 nm (850 nm+28 nm). This prediction assumes analogous pigment-protein dynamics between the bchla and bchlb variants. The PLQY for LvP-bchlb was expected to be low (<5%, similar to LvP-bchla's 3.8%) due to enhanced non-radiative decay pathways in the NIR, favoring photoacoustic signal generation over fluorescence, which could be validated using NIR-optimized spectrofluorometers.

### 2. Photoacoustic Response Across a Range of Excitation Wavelengths

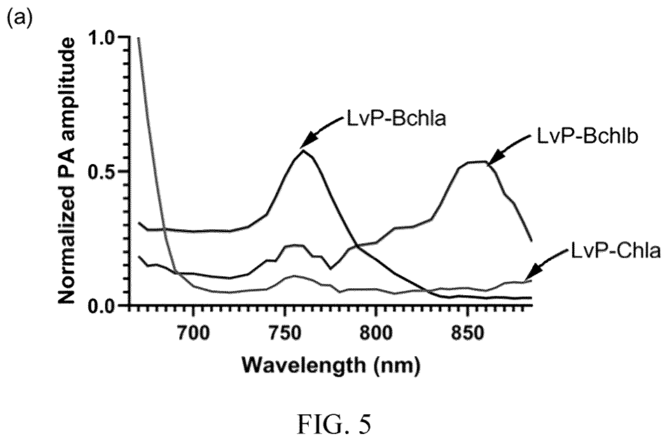

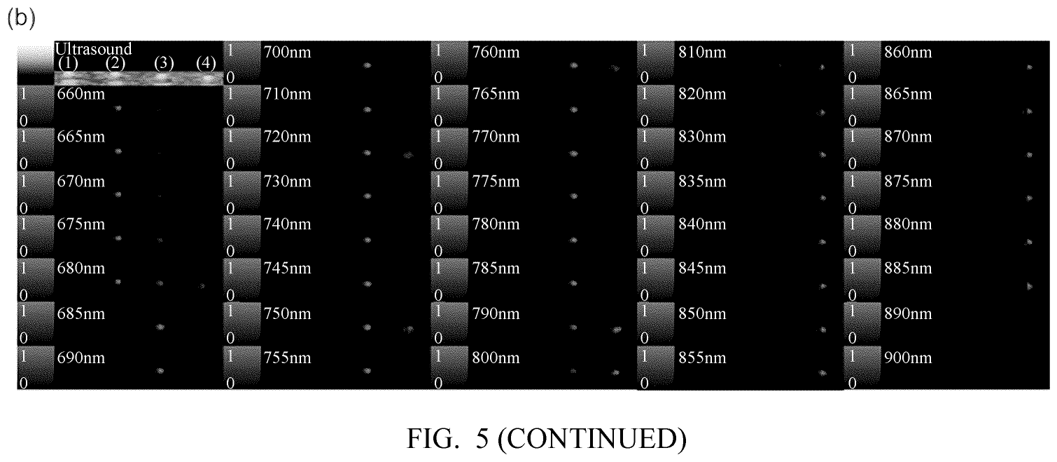

The PA response of the three LvP complexes (LvP-chla, LvP-bchla, and LvP-bchlb) was evaluated under excitation wavelengths ranging from 660 nm to 900 nm. FIG. 5 illustrates the variation in PA amplitude for each complex as a function of excitation wavelength. The results confirmed the optimal excitation wavelengths for each complex based on their respective absorption maxima, further highlighting their versatility for multi-wavelength PA imaging. Moreover, the efficiency of PA wave generation follows the extinction coefficients observed in FIG. 3, where bacteriochlorophyll complexes have a lower efficiency than chlorophyll a, which has a two-fold higher extinction coefficient. The full-width half maximum (FWHM) for bacteriochlorophyll a and b was measured at +30 nm from the Qy peaks, respectively, and +20 nm from the Qy peak for chlorophyll a. The FWHM value can be used for spectroscopic PAI for all three variants of the contrast agents, which are also orthogonal to each other, opening possibilities for PAI with multiple targets.

3. Concentration-Dependent PA Signal Generation and Spectroscopic Differentiation from Endogenous Absorbers

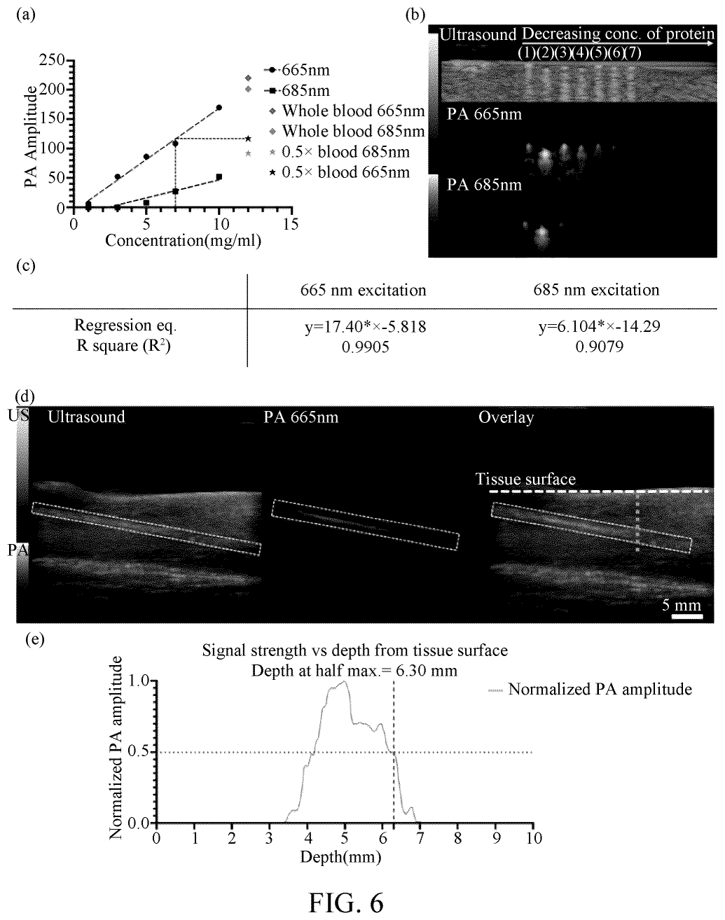

Phantom studies were conducted to evaluate the PA signal generation efficiency of LvP-chla (FIG. 6). Solutions of LvP-chla with increasing concentrations (from 1 mg/mL to 10 mg/mL) were prepared and imaged using a PAI system. LvP-chla successfully generated PA signals across concentrations ranging from 1 mg/mL to 10 mg/mL under 665 nm excitation. FIG. 6a demonstrates a linear relationship between the PA signal amplitude and the concentration of LvP-chla, indicating effective solubilization and prevention of aggregation.

Moreover, the efficiency of generating PA signals was compared between two close excitation wavelengths, 665 nm and 685 nm. The selection of 665 nm and 685 nm excitation wavelengths leverages the maximum absorption peak and halves the absorption of the LvP-chla spectral profile while enabling differential imaging with minimal penetration depth variations and consistent laser energy output owing to their narrow separation. A linear relationship between PA signal amplitude and LvP-chla concentration was observed for both 665 nm and 685 nm excitations, with whole blood and 0.5× blood included as controls (FIG. 6a-b). The linear regression analysis yielded high coefficients of determination (R²=0.9905 and 0.9079 for 665 nm and 685 nm excitation, respectively) with regression equations of y=17.40*x−5.818 and y=6.104*x−14.29, demonstrating robust concentration-dependent PA signal generation (FIG. 6c).

Despite the mere 20 nm difference, the signal amplitude at 685 nm decreased to one-third of that at 665 nm, which is at the absorption maximum. This significant reduction highlights the sensitivity of PA signal generation to excitation wavelength. As the concentration of the contrast agent increases, the viscosity increases and harms the injectability of the exogenous contrast agent. The rapid drop in PA signal generation with a slight change in excitation wavelength solves the challenges associated with high protein concentrations. By utilizing spectroscopic PAI, it is possible to overcome the challenges associated with high protein concentrations. This technique allows for the differentiation of the contrast agent from background signals, even at lower concentrations, by exploiting the specific spectral signatures of LvP-chla.

For the penetration depth study, 5 mg/mL LvP-chla was selected as it provides sufficient signal strength while maintaining low viscosity and cytotoxicity, making it suitable for subsequent intratumoral injection. The penetration depth characteristics were evaluated using chicken breast tissue as a biological scattering medium (FIG. 6d). Quantitative signal analysis revealed the half-maximum amplitude at 6.30 mm depth (FIG. 6e), indicating promising potential for deep-tissue imaging applications.

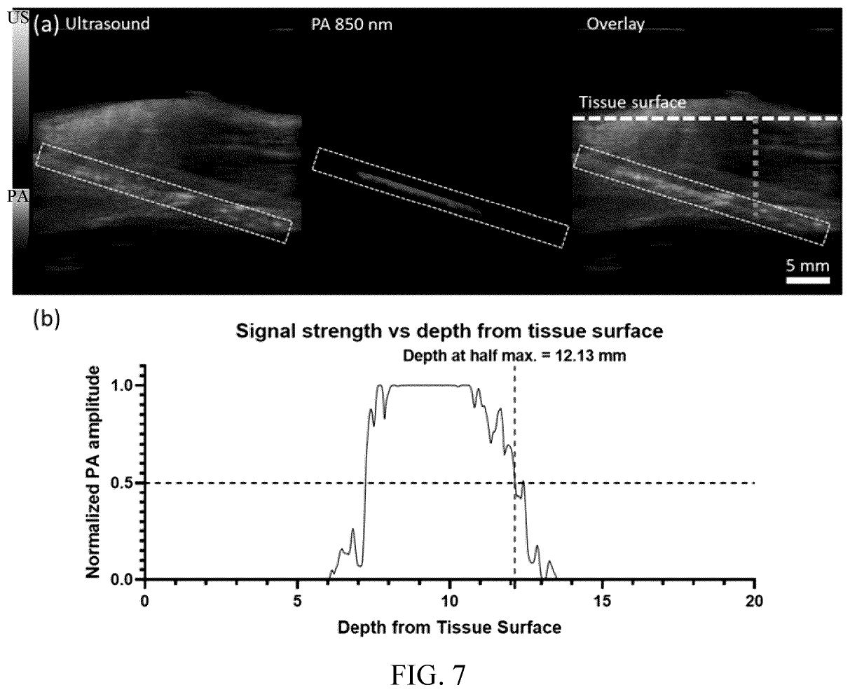

A penetration depth study was also conducted for LvP-bchlb (FIG. 7) under experimental conditions substantially identical to those employed for LvP-chla.

These findings underscore the potential of the three LvP complexes as an efficient and versatile PA contrast agent capable of delivering strong signals at optimal wavelengths while mitigating issues related to high-concentration formulations.

### 4. In Vitro Evaluation of Suitable LvP-Chla Concentration for PAI

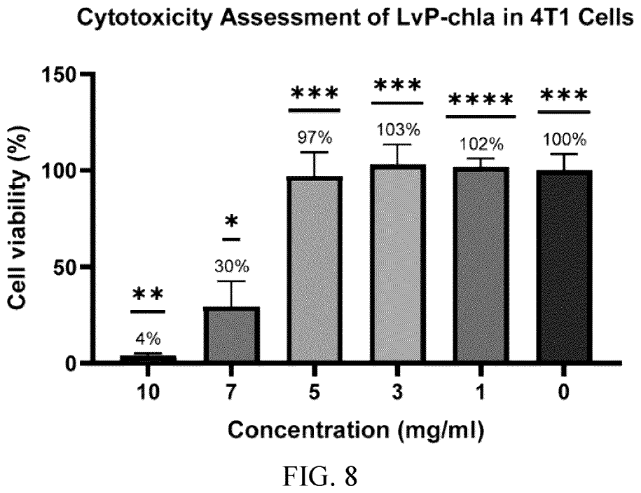

In order to evaluate the tolerable dosage for in vivo imaging, the cytotoxic effects of LvP-chla on 4T1 cells were evaluated using the MTT assay. A clear concentration-dependent cytotoxic response was observed after 12-h exposure to LvP-chla (FIG. 8). LvP-chla treatments at lower concentrations (1, 3, and 5 mg/mL) showed no significant cytotoxic effects, maintaining cell viability close to the untreated control level. Conversely, higher LvP-chla concentrations (7 and 10 mg/mL) significantly reduced cell viability compared to the untreated control (p<0.05 and p<0.01, respectively). These results demonstrate that LvP-chla concentrations at or above 7 mg/mL exert pronounced cytotoxic effects on 4T1 cells, while 5 mg/mL is a safe and effective dose for in vivo studies.

### 5. In Vivo Application of LvP-Chla as a PA Contrast Agent

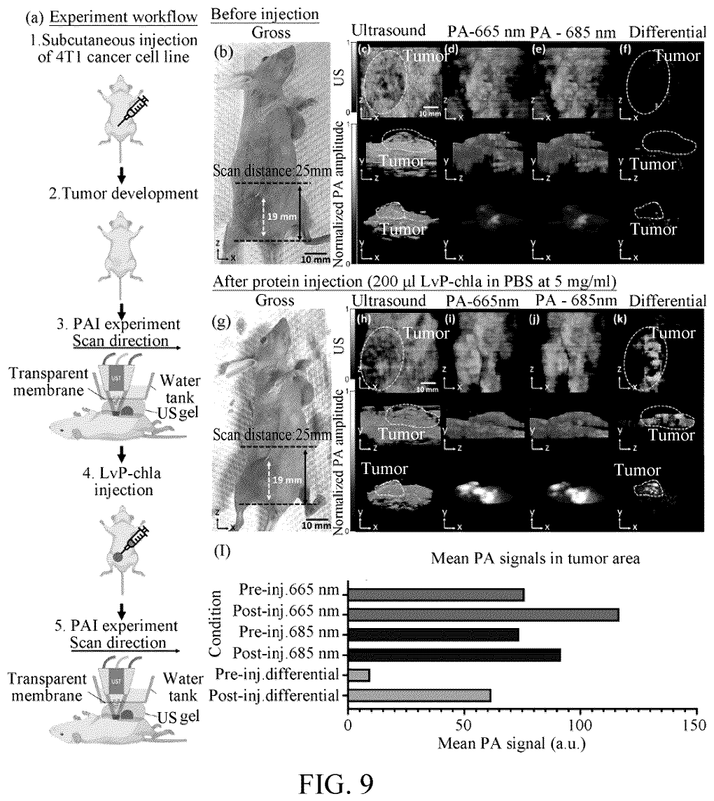

Following successful in vitro validation, the efficacy of LvP-chla as an in vivo PA contrast agent was evaluated using a nude mouse 4T1 allograft model. Utilizing the optimized 5 mg/mL concentration from the cytotoxicity test, 200 μL of LvP-chla was administered intratumorally, followed by dual-wavelength spectroscopic PAI at 665 nm and 685 nm (FIG. 9). The experimental workflow is illustrated in FIG. 9a. Baseline imaging prior to LvP-chla administration revealed typical endogenous PA signals at both wavelengths (FIG. 9d,e) with negligible contrast in the differential images at the tumor site (FIG. 9f).

The concurrent ultrasound imaging (FIG. 9c) provides essential anatomical context, enabling precise tumor localization and structural information while demonstrating the system's dual-modality capability. Post-administration imaging (FIG. 9g-k) demonstrated pronounced PA signal enhancement localized within the tumor region, with ultrasound images (FIG. 9h) continuing to provide anatomical guidance for accurate spatial registration of the PA image. The differential imaging approach (FIG. 9k) particularly enhanced visualization of the contrast agent distribution by effectively eliminating background signals while maintaining the structural context provided by ultrasound imaging. There is a 6.52-fold increase in mean PA signal strength in the tumor area, as shown in FIG. 9l in the differential image, driven by LvP-chla's strong absorption at 665 nm (59,716 M⁻¹ cm⁻¹) and effective background subtraction, compared to a 1.54-fold increase at 665 nm and a 1.24-fold increase at 685 nm.

These findings highlight the capability of LvP-chla, in conjunction with dual-wavelength differential PAI, to provide high-contrast molecular information overlaid on ultrasound images, representing a significant advancement in dual-modality molecular imaging that combines the specificity of PAI with the anatomical information of ultrasound.

### 6. In Vivo Clearance of LvP-Chla

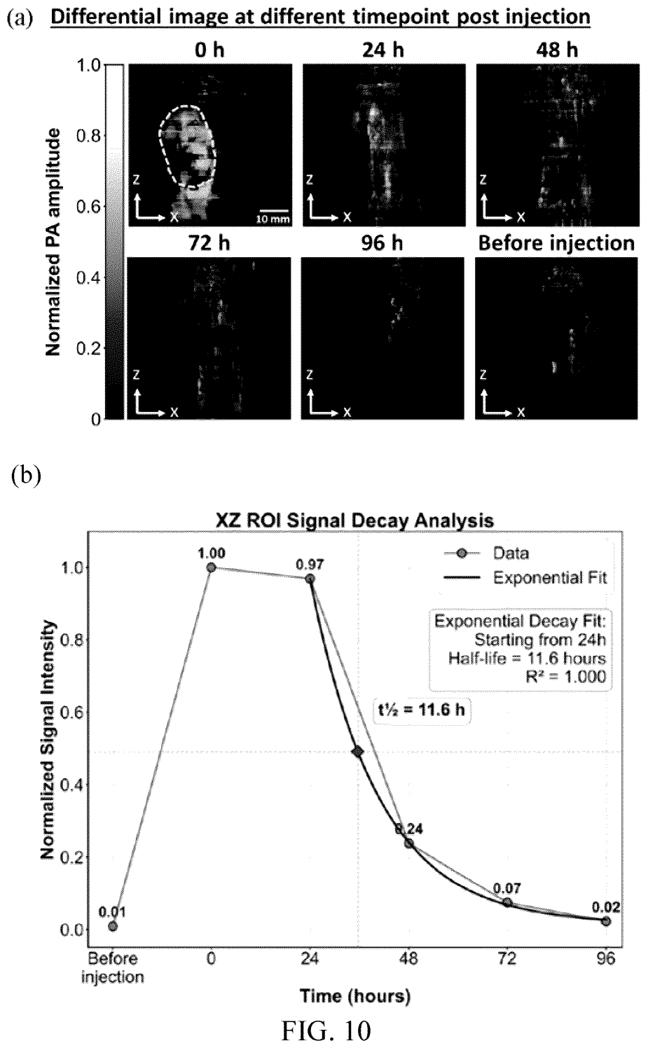

To assess in vivo clearance, longitudinal PAI was conducted after LvP-chla injection in nude mice bearing 4T1 tumors, using pre-injection PA signals as baseline references. Differential PA images acquired over 96 h clearly showed a gradual decrease in signal intensity (FIG. 10). Exponential decay analysis of normalized PA signals revealed signal half-lives (t½) of approximately 11.6 h (R²=1.000) with signal plateauing 24 h post-injection. Importantly, PA signals returned to baseline within 96 h post-injection, indicating minimal long-term retention of LvP-chla in tumor tissue.

### 7. In Vivo Toxicity Evaluation

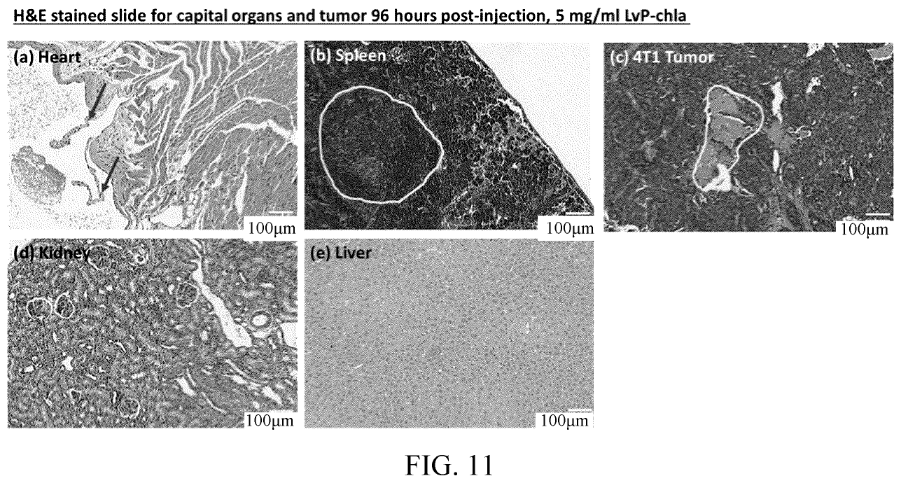

For the toxicity check of LvP-chla, the mice exhibited no signs of distress throughout the observation period, and tumor size remained unchanged. Histological analysis of vital organs (FIG. 11) reveals no cellular damage or abnormality in the heart (FIG. 11a), kidneys (FIG. 11d), or liver (FIG. 11e) following LvP-chla administration.

While splenomegaly was observed with an increase in white blood cells in the spleen, this is likely attributed to an immune response towards tumor growth rather than LvP-chla injection, as the normal white pulp is observed in FIG. 11b.

Overall, these results demonstrate that LvP-chla exhibits no observable toxicity, enabling safe and longitudinal monitoring of targeted biomarkers in deep tissues.

What is claimed is:

1. A method for photoacoustic imaging a subject, the method comprising:
(a) administering an effective amount of a water-soluble chlorophyll-binding protein (WSCP)-chlorophyll complex at a site of the subject, wherein the WSCP-chlorophyll complex comprises a Lepidium virginicum WSCP (LvP) and a chlorophyll selected from the group consisting of chlorophyll a, bacteriochlorophyll a, and bacteriochlorophyll b;
(b) irradiating the subject with light thereby inducing a photoacoustic signal from the WSCP-chlorophyll complex;
(c) detecting the photoacoustic signal; and
(d) generating a photoacoustic image of the site based on the detected photoacoustic signal.

2. The method of claim 1, wherein the light is pulsed light having a wavelength in the range of 650 nm to 900 nm.

3. The method of claim 1, wherein the WSCP-chlorophyll complex exhibits a Qy absorption peak at a wavelength of the pulsed light in the range of about 665 nm to 850 nm and a Qx absorption peak at a wavelength of the pulsed light in the range of about 578 nm to 592 nm.

4. The method of claim 2, wherein the WSCP-chlorophyll complex exhibits: a Qy absorption peak at a wavelength of the pulsed light of about 665 nm; a Qy absorption peak at a wavelength of the pulsed light of about 765 nm, and a Qx absorption peak at a wavelength of the pulsed light of about 578 nm; or a Qy absorption peak at a wavelength of the pulsed light of about 850 nm, and a Qx absorption peak at a wavelength of the pulsed light of about 592 nm.

5. The method of claim 1, wherein the chlorophyll a is Spinacia oleracea chlorophyll a, the bacteriochlorophyll a is Rhodospirillum rubrum bacteriochlorophyll a, or the bacteriochlorophyll b is Blastochloris viridis bacteriochlorophyll b.

6. The method of claim 2, wherein the method further comprises:
(b1) irradiating the subject with a first pulsed light at a first wavelength to induce a first photoacoustic signal from the WSCP-chlorophyll complex;
(b2) irradiating the subject with a second pulsed light at a second wavelength that is different from the first wavelength to induce a second photoacoustic signal from the WSCP-chlorophyll complex;
(c1) detecting the first photoacoustic signal and the second photoacoustic signal; and
(d1) generating a differential photoacoustic image of the site based on the detected first photoacoustic signal and the detected second photoacoustic signal.

7. The method of claim 6, wherein the first and second wavelengths differs in a range from about 20-235 nm, or from about 20-50 nm; or
wherein the WSCP-chlorophyll complex exhibits a Qy absorption peak at one of the first and second wavelengths, and a reduced or negligible optical absorption at the other of the first and second wavelengths.

8. The method of claim 6, wherein the first and second wavelengths, or the second and first wavelengths, are:
in the range of about 650 nm to 850 nm and about 685 nm to 900 nm, respectively;
about 665 nm and about 685 nm, respectively;
about 765 nm and about 800 nm, respectively; or
about 850 nm and about 890 nm, respectively.

9. The method of claim 6, wherein generating the differential photoacoustic image comprises subtracting the second photoacoustic signal from the first photoacoustic signal, or subtracting the first photoacoustic signal from the second photoacoustic signal.

10. The method of claim 2, further comprising:
(b3) irradiating the subject with pulsed light prior to administration of the WSCP-chlorophyll complex to induce a background photoacoustic signal from endogenous chromophores at the site;
(c3) detecting the background photoacoustic signal; and
(d3) generating a differential photoacoustic image of the site based on the detected background photoacoustic signal and the photoacoustic signal detected after administration of the WSCP-chlorophyll complex.

11. The method of claim 10, wherein the pulsed light used to iridate the subject before and after administration of the WSCP-chlorophyll complex is of the same wavelength; and
wherein the wavelength is within a range of about 650-850 nm.

12. The method of claim 10, wherein the wavelength is about 665 nm, about 765 nm, or about 850 nm.

13. The method of claim 10, wherein generating the differential photoacoustic image comprises subtracting the background photoacoustic signal from the photoacoustic signal detected after administration of the WSCP-chlorophyll complex.

14. The method of claim 1, wherein the method comprising:
(a4) administering an effective amount of two or more water-soluble chlorophyll-binding protein (WSCP)-chlorophyll complexes at two or more target sites of the subject, wherein each WSCP-chlorophyll complexes comprise a Lepidium virginicum WSCP (LvP) and a chlorophyll selected from the group consisting of chlorophyll a, bacteriochlorophyll a, and bacteriochlorophyll b;
(b4) irradiating the subject with pulsed light at two or more excitation wavelengths thereby inducing two or more photoacoustic signals from the respective WSCP-chlorophyll complexes;
(c4) detecting photoacoustic signals; and
(d4) generating a composite photoacoustic image that spatially resolves the different target sites based on the detected photoacoustic signals.

15. The method of claim 14, wherein the two or more excitation wavelengths correspond to the absorption maxima of the respective WSCP-chlorophyll complexes and are in the range of about 665 nm to 850 nm.

16. The method of claim 14, wherein the chlorophyll comprises any two or all of Spinacia oleracea chlorophyll a, Rhodospirillum rubrum bacteriochlorophyll a, and Blastochloris viridis bacteriochlorophyll b; or
the excitation wavelengths comprise any two or all of the following: about 665 nm, about 765 nm, and about 850 nm.

17. The method of claim 1, wherein the subject is a human, a non-human primate, a rodent, a canine, a feline, a bovine, or an equine.

18. A method for photoacoustic imaging, the method comprising:
providing an effective amount of a water-soluble chlorophyll-binding protein (WSCP)-chlorophyll complex comprising a Lepidium virginicum WSCP (LvP) and a chlorophyll selected from the group consisting of chlorophyll a, bacteriochlorophyll a, and bacteriochlorophyll b;
irradiating the WSCP-chlorophyll complex with pulsed light having a wavelength in the range of 650 nm to 900 nm thereby inducing a photoacoustic signal;
detecting the photoacoustic signal; and
generating a photoacoustic image based on the detected photoacoustic signal.

19. The method of claim 18, wherein the chlorophyll a is Spinacia oleracea chlorophyll a, the bacteriochlorophyll a is Rhodospirillum rubrum bacteriochlorophyll a, or the bacteriochlorophyll b is Blastochloris viridis bacteriochlorophyll b.

20. A water-soluble chlorophyll-binding protein (WSCP)-chlorophyll complex, comprising a Lepidium virginicum WSCP (LvP) and Rhodospirillum rubrum bacteriochlorophyll a, or Blastochloris viridis bacteriochlorophyll b.
